(19) United States
(12) Patent Application Publication
(10) Pub. No.: US 2004/0180915 A1
(43) Pub. Date: Sep. 16, 2004

(54) Methadone-containing compositions for parenteral administration and method of use thereof

(75) Inventors: Gilbert R. Gonzales, New York, NY (US); James L. Young, West Chester, OH (US); Ralph A. Heasley, Union, KY (US); Paolo L. Manfredi, New York, NY (US)

Correspondence Address: WOOD, HERRON & EVANS, LLP, 2700 CAREW TOWER, 441 VINE STREET, CINCINNATI, OH, 45202, US

(73) Assignee: Xanodyne Pharmacal, Inc., Florence, KY

(21) Appl. No.: 10/389,476

(22) Filed: Mar. 14, 2003

(51) Int. Cl. A61K 31/485

(52) U.S. Cl. 514/282

(57) ABSTRACT

Compositions and methods for parenterally administering methadone. The methadone composition may be administered in a sterile, preservative-free formulation, or in a formulation including a chlorobutanol-free, government approved preservative. The methadone formulations are administered parenterally, such as intravenously or topically, for many applications including the treatment of pain resulting from chronic disorders, cancer, acute symptoms and the like.

### BACKGROUND OF THE INVENTION

I. Field of the Invention

The present invention generally relates to parenterally administered pharmaceutical compositions including methadone, and in particular, to compositions including a combination of methadone and a preservative.

II. Description of the Prior Art

Methadone is an opioid analgesic, a mu receptor agonist, which is commonly compared with the standard opioid analgesic, morphine, in terms of efficacy and application in single doses. Acutely, the potency and efficacy of methadone are at least equal to those of morphine. However, methadone has many benefits over morphine, as well as over other administered opioid analgesics. Methadone's pharmaco-dynamic profile is longer acting than morphine's, allowing for twice or three times a day dosing. Tolerance develops to the analgesic effects more slowly with methadone than with morphine. In addition, methadone has an extended duration of action in suppressing withdrawal symptoms in physically dependent individuals, and in particular, the withdrawal signs and symptoms occurring after abrupt discontinuance of methadone are milder than those of morphine. Further, methadone has a tendency to exhibit persistent effects with repeated administration. These properties make methadone a useful drug and, therefore, one that is commonly administered.

One outstanding property of methadone is its effective analgesic activity. Thus, methadone is administered for the treatment of pain, especially pain refractory to other medications. Particularly, methadone is effective for the relief and management of severe, constant pain, such as chronic cancer pain. However, methadone's analgesic activity is not so limited and methadone is known to be effective for many other types of pain as well, including neuropathic pain. In adults, methadone is typically administered in doses ranging from about 2 mg to about 10 mg, depending upon a number of factors including the age, weight, and the purpose of administration. Methadone is also administered for the treatment of opioid abstinence syndromes, and for the treatment, detoxification, and maintenance of a chronic relapsing heroin addict. For detoxification of a heroin-dependent addict, low doses of methadone (5-10 mg orally) are given 2 or 3 times daily for 2 or 3 days. Methadone also has been found to be an effective antitussive agent. Methadone does, however, produce some side effects, including respiratory depression, myosis, and effects on the secretion of pituitary hormones that are qualitatively similar to the effects of morphine.

Methadone hydrochloride, USP, is a bitter white powder soluble in water and ethanol, and is generally administered both orally and parenterally. In the United States, special controls of methadone have been enacted in an effort to prevent its unregulated large-scale use in the treatment of opioid addiction. Accordingly, specialized dosage forms used in opioid addiction include tablets containing 2.5, 5, 10, or 40 mg of the drug. Currently available formulations of methadone include methadone hydrochloride tablets, USP, and are available for oral use in a 5 and 10 mg amounts.

Orally administered methadone is well absorbed from the gastrointestinal tract and can be detected in the blood plasma within 30 minutes after ingestion. However, orally ingested methadone undergoes extensive metabolism in the liver. The major metabolites, resulting from N-demethylation and cyclization to form pyrrolidine and pyrroline, are excreted in the urine and the bile along with small amounts of unchanged drug. Orally administered methadone results in a half-life of a single methadone dose of approximately 15 hours, and therefore, requires a large dose or multiple daily doses to achieve the desired effect.

Thus, methadone is often administered parenterally by routes other than oral ingestion. Appreciable concentrations of methadone have been found in the plasma within 10 minutes after subcutaneous injection. Further, peak concentrations of the methadone occur in the brain within 1 to 2 hours after subcutaneous or intramuscular administration, and this correlates well with the intensity and duration of methadone's analgesic effect. In terms of total analgesic effects, methadone given orally is about 50% as effective as the same dose administered intramuscularly. To this end, it is desirable to develop a useful formulation of methadone for parenteral administration to a person in need thereof.

Parenteral methadone has been approved by the FDA for intramuscular administration only and the only commercial formulation approved and available for use is a 20 ml multi-dose vial containing a solution of methadone (10 mg/ml) and 0.5% chlorobutanol (5 mg/ml), as a preservative. Unfortunately, however, it is believed that the chlorobutanol, present in the solution at concentrations of only about 0.5%, may be linked to the cause of death of patients who suffered from stage 1V cancer and received the FDA approved methadone solution via intravenous injection (IV). Particularly, it is believed that the preservative chlorobutanol in the IV methadone solution is a probable and likely cause of QTc interval prolongation in the patients. In support, chlorobutanol has been shown to increase action potential duration, lower conduction velocity, and induced more automoticity in amphibian heart cells (Hermes Mayer and Aprig Leono, 1976). Further, chlorobutanol has a very long half-life (over 10 days), which may allow for significant accumulation in the plasma. Thus, in the United States, the only available parenterally administrable methadone is a formulation that has shown significant potential for cardiotoxicity.

Accordingly, it is desirable to provide a parenterally administrable formulation of methadone that will effectively treat patients with severe pain and pain refractory to other opioids while not exhibiting cardiotoxic effects. It is also desirable to provide a formulation that is government approved. It is further desirable to include in the methadone formulation a preservative that is government approved.

### SUMMARY OF THE INVENTION

The present invention provides compositions, for parenteral administration, comprising methadone and methods of administering the same for the treatment of refractory pain while addressing the weaknesses and drawbacks of the prior art parenterally administrable methadone formulation. Particularly, the present invention eliminates the need to include chlorobutanol in the composition. To this end, and in accordance with the principles of the present invention, there is provided a pharmaceutical composition comprising methadone and a chlorobutanol-free preservative, in a parenterally administrable formulation. The formulation may optionally be sterilized to enhance compliance with government regulations and to secure approval for use.

The composition should include methadone in concentrations and amounts approved by the government for use. Thus, the methadone should have a concentration no greater than about 50 mg/ml in the formulation, and should not be dosed in an amount that may be toxic. It is useful to include smaller concentrations or more dilute formulations for topical and subcutaneous use. For example, for topical administration, the methadone may have a concentration in the range from about 0.01 mg/ml to about 20 mg/ml in the formulation.

The preservative is chlorobutanol-free and should be present in government approved amounts. As a result, the methadone formulations of the present invention should not have the cardiotoxicity of the prior art formulation. To this end, proven preservatives which may be suitable for inclusion in the present compositions include, without limitation, compounds of the following classes: parabens, aromatic alcohols, cresols, acetates, borates, nitrates, acids, and combinations thereof. By way of example, antimicrobial preservatives including benzalkonium chloride, benzethonium chloride, benzyl alcohol, phenethyl alcohol, phenoxyethanol, meta-cresol, chlorocresol, phenol, chlorhexidine, methylparaben, propylparaben, butylparaben, phenylmercuric acetate, phenylmercuric borate, phenylmercuric nitrate, thimerosal, benzoic acid, sorbic acid, cetrimide, myristyl gamma-picolinium chloride, and combinations thereof, are suitable.

In another aspect of the present invention, sterile methadone-containing formulations may be prepared without a preservative. Sterilization eliminates the need for a preservative while allowing for government approval for use. Suitable sterilizing techniques include subjecting the formulation to sterile filtration, irradiation, heat, autoclaving or a combination thereof. Such sterile formulations are useful for many applications, as discussed herein.

Parenteral administration is, by definition, administration by means other than oral ingestion. Therefore, the methadone-containing formulation may be administered in a variety of non-ingestion methods including, for example, intravenous administration, intramuscular administration, and subcutaneous injection. The formulation may also be topically administered to a membrane, such as a mucous membrane including a nasal membrane, a buccal membrane, a vaginal membrane, a rectal membrane, and an ocular membrane, for the treatment of pain therein. The formulations should be administered in amounts effective for treating pain generally attributable to cancer, chronic disorders, acute symptoms, neuropathic disorders, or a combination thereof. Acute pains include, for example, somatic pain, while neuropathic pain includes, for example, autonomic nervous system pain and central pain.

The formulation should be suitable for the desired application and government-approved. Suitable formulations include, for example, a solution, a paste, a powder, an emulsion and a suspension. Solutions may be readily prepared by dissolving the methadone and chlorobutanol-free preservative, where desired, in one or more suitable diluents such as water, saline solution, and alcohol. Powders may be prepared for dilution or dissolution just prior to use. Pastes, emulsions, and suspensions may be prepared with a suitable diluent as well. Conventional pharmaceutically accepted methods for preparing the formulations are suitable.

Thus, there are provided compositions including methadone which may be government approved for parenteral administration to a patient, by either sterilization of the formulation and/or inclusion of a chlorobutanol-free preservative. To this end, the present invention addresses the cardiotoxic effects of chlorobutanol included within the prior art parenterally administrable methadone formulation. These and other benefits and advantages of the present invention shall be made apparent from the accompanying detailed description below.

### DETAILED DESCRIPTION OF THE INVENTION

The present invention provides compositions including methadone, in parenterally administrable, chlorobutanol-free formulations that address the weaknesses and drawbacks of the comparable prior art methadone formulation. The methadone formulation may be sterilized, and/or include therein a chlorobutanol-free preservative, to meet government standards for patient use while addressing the cardiotoxicity of chlorobutanol, present in the parenterally administered methadone formulation of the prior art. Methadone is a proven opioid analgesic, useful in patients in treating pain refractory to morphine. Methadone is also useful for treating chronic pain, cancer pain, acute pain including, without limitation, somatic pain, neuropathic pain including autonomic nervous system pain and central pain, and combinations thereof in the body. Accordingly, the present methadone comprising compositions and formulations may be administered for such use. In addition, the formulations may be used for treating pathological itching.

The term “parenteral”, as used herein with respect to the mode of administration, includes all modes of administration other than oral ingestion. For example, parenteral administration includes topical administration where the methadone formulation is topically applied onto a membrane of the patient. For example, the membrane may be a mucosal membrane or simply the patient's skin or epidermis. In one embodiment of the present invention, the methadone formulation is topically administered to the mucosal membrane for treatment of pain. In another embodiment, the mucous membrane treated is a nasal, buccal, vaginal, or rectal membrane. Further, the methadone formulation may be administered, in a suitable form, onto the surface of an eye for treatment of ocular pain. For example, the methadone may be formulated as an eye-drop solution which may be applied to the eye as needed. Additional examples of “parenteral” administration include subcutaneous or intramuscular administration where the methadone formulation is generally a solution that is formulated to enter the body of the patient either through an injection via syringe or intravenously, through an IV tube, modes commonly used in hospitals. The term, “parenterally administering”, as used herein, is construed broadly and refers to not only to the person(s) actually performing the administration, but also to the person(s) directing the administration of the methadone-containing formulation to the patient.

The amount of methadone included in the present compositions may vary, but should remain within the concentrations and dosage guidelines provided by Food and Drug Administration regulations and generally accepted clinical practices. As such, the methadone should be administered in a dosage amount which is effective for treatment of opioid abuse, pain, or other application. As previously discussed, the dosage for administration generally depends upon many factors, such as the age, size, weight, gender and prior medical history of the patient, as well as the purpose of administration. For example, suitable parenteral doses of methadone for use in opioid naive patients include about 0.5 mg to about 2 mg. This dosage range may increase with practically no ceiling for treatment of opioid tolerant patients. Higher ranges may be useful on patients that are treated and who are opioid tolerant having been previously treated with high doses of morphine and/or other opioids and were converted to parenteral administration of methadone.

The amount of methadone included in the formulation used primarily for the treatment of pain and pain refractory to other opioids may also vary, but it's generally safer, from a tolerance and dependence standpoint, to include lower concentrations. In one embodiment, a formulation comprising methadone in a concentration range from about 0.005 mg/ml to about 50 mg/ml of diluent may be administered for treatment. In another embodiment, a formulation comprising methadone in a concentration range from about 0.01 mg/ml to about 10 mg/ml of diluent may be administered for treating ocular pain. In another embodiment, a formulation comprising methadone in a concentration range from about 0.1 mg/ml to about 20 mg/ml of a diluent may be topically administered to a mucous membrane for treating pain. In another embodiment, a formulation comprising methadone in a concentration range from about 0.005 mg/ml to about 20 mg/ml of a diluent may be administered via the epidermis for treating pain. In yet another embodiment, a formulation comprising methadone in a concentration range from about 0.005 mg/ml to about 0.1 mg/ml of diluent may be administered intrathecally (to the spinal fluid) for treating pain. Amounts effective for treating pathological itching will vary in accordance with the dependency factors discussed herein, and will generally fall within the broad concentration range described above.

In one aspect of the present invention, the methadone composition or formulation may be sterilized in order to comply with government regulations and standards, particularly those set forth by the Food and Drug Administration (FDA) and the United States Pharmacopiea (USP). Sterilization renders the formulation free from viable pathogens and ensures a low endotoxin level in compliance with government standards. Sterilization may be accomplished by conventional methods and techniques. For example, solution formulations may be sterilized by sterile filtration, irradiation, heating, or autoclaving of the solution. Other liquid or semi-solid formulations, such as suspensions, pastes, creams, emulsions, and the like, may also be sterilized by conventional methods including, without limitation, irradiation, heating, autoclaving, or a combination thereof. Sterility is a Federal requirement for all parenterally administered formulations.

In order to secure government approval for parenteral administration, the present composition may further include a preservative. The term “preservative”, as used herein, is intended to have its common pharmaceutical meaning, that is, a substance that prevents or inhibits microbial growth and may be added to pharmaceutical preparations for this purpose to avoid consequent spoilage of the preparations and formulations by microorganisms. Accordingly, the addition of a preservative may insure that multiple dose vials remain sterile during their use by preventing the growth and survival of microorganisms introduced into the multi-dose vial during use. Thus, in another aspect of the invention, the present composition includes a preservative to maintain sterility of multi-dose formulations during use, thereby alleviating the need for sterilizing the formulation by a technique described above.

The only methadone formulation currently approved by the FDA for parenteral administration, however, includes chlorobutanol, in small quantities (about 0.5%), as a preservative. The chlorobutanol in the methadone solution may to be related to deaths of patients who were parenterally administered methadone by injection for treating pain related to stage IV cancer. Particularly, cardiotoxic effects exhibited by QTc interval prolongation in the patients may have been caused by the chorobutanol in the solution. The cardiotoxic effects of the present parenteral methadone formulation is supported by studies conducted by the Applicants. Applicants' carefully monitored the ECG's of patients receiving IV methadone, including chlorobutanol even at levels of about 0.5% by volume. Applicants' also performed a comparison study involving hundreds of EKG's performed on patients treated with IV methadone versus the EKG's of patients not being treated with the IV methadone. Applicants found that the QTc intervals of patients on and off methadone were 43.2 milliseconds (+/−7.7) greater for the patients treated with the IV methadone versus those without. Comparable studies utilizing orally ingested methadone in a formulation without chlorobutanol did not show a similar prolongation in QTc interval times. Moreover, QTc prolongation reversed when the methadone was switched from an IV route to an oral route in at least one patient, suggesting that factors other than the methadone itself may be primarily implicated in causing QTc prolongation. A similar study was performed with the use of an IV morphine solution formulated with a non-chlorobutanol-containing preservative. The patients on IV morphine did not show such a discrepancy in the QTc interval times. Equipped with the information that chlorobutanol has been shown to increase action potential duration, lower conduction velocity, and induced automoticity in amphibian heart cells (Hermes Mayer and Aprig Leono, 1976), and that chlorobutanol has a very long half-life (over 10 days) which may allow for significant accumulation in the plasma, Applicants' arrived at the conclusion that the preservative, chlorobutanol, in the IV administered methadone formulation is the probable and likely cause of the QTc time prolongation and cardiotoxicity.

To this end, and in accordance with the principles of the present invention, the preservative included in the present methadone composition will be free of chlorobutanol. By elimination of this cardiotoxic agent, the present compositions address the side effects of chlorobutanol suffered in the prior art formulation. Other known and government-approved preservatives (approved for use in other applications) may serve the same function and purpose of chlorobutanol without posing the risk of cardiotoxic side effects. The term “government approved”, as used herein, is intended to generally refer to approval of the preservative by the United States government or by governments outside the United States. It should be appreciated by those skilled in the art that the government approval for use of a preservative in a formulation is valid only in that country whose government gave approval. Many organic compounds belonging to different compound classes have been approved for use as a preservative. For example, parabens, including methylparaben, propylparaben, and butylparaben have been approved and used in formulations and medications. Various alcohols, and in particular aromatic alcohols such as benzyl alcohol, phenethyl alcohol, and phenoxyethanol, as well as cresols including meta-cresol, chlorocresol, and phenol have also been approved for use as preservatives in medications. Additional examples include, without limitation, acetates such as phenylmercuric acetate, borates such as phenylmercuric borate, nitrates such as phenylmercuric nitrate, and carboxylic acids such as benzoic acid, sorbic acid and proprionic acid and their salts, all of which have been approved and are suitable for use as a preservative in the present invention. In addition to the above, further suitable preservatives include benzalkonium chloride, benzethonium chloride, thimerosal, cetrimide, myristyl gamma-picolinium chloride, and any combination of the preservatives described above. It should be understood that regardless of the number or combination of preservatives included, the present composition and formulation will not include chlorobutanol in any form, amount, and for any purpose.

The effectiveness of an antimicrobial preservative, such as those described above, in a formulation is generally demonstrated by using a specific test. All of the tests involve mixing the preserved formulations with standard culture of gram-positive and gram-negative bacteria, yeast and molds, and counting the number of viable microorganisms remaining at different timepoints after inoculation. The preservative is deemed to be effective in a formulation if the concentration of each test microorganism remains at or below stipulated levels determined by a government, and hence the approval for use over a given shelf life. Thus, to effectively preserve a formulation, the preservative must have a broad spectrum of activity as to range of microorganisms. In this respect, the USP generally requires demonstration that any test microorganisms inoculated in the formulation are cleared from the formulation in a prescribed fashion over the period of the test. In this respect, the United States guidelines and regulatory practices are comparable to European guidelines. For example, both the USP and the European Pharmacopeia (EP) requires preservative-containing formulations to be completely microorganism-free, i.e. sterile, 100% free of microorganism for any period of time. EP and USP standards require that preservatives in multi-dose containers are formulated in such a manner to reduce microorganisms introduced into the multi-dose containers by a specified amount in a specified time period.

The major criteria that should be considered in selecting a preservative are as follows: It should be effective against a wide spectrum of microorganisms, stable for its shelf-life, non-toxic, non-sensitizing, compatible with the ingredients in the dosage form, inexpensive, and essentially relatively free of objectionable taste and odor. In addition to the above, there are a number of specific factors which should be taken into account when a preservative is selected: 1) The site of use, for example, is the formulation being used externally, internally, or for ophthalmic reasons? 2) The pH of the liquid, as it may affect both the ionization of the preservative and its stability. 3) The solvent, as this will affect the solubility of the preservative. 4) Partitioning into an oil phase of an emulsion, thereby reducing the concentration in the aqueous phase where preservative action takes place. 5) Adsorption into or onto the solid phase of the suspension, thereby reducing the concentration of the aqueous phase. 6) Processing and packaging variables such as heat, order of addition of the ingredients, stirring or container materials. 7) Type of dosage form, e.g., solution, emulsion, or suspension, as discussed herein.

With this in mind, the concentration of the preservative in the formulation may vary, so long as it is effective to render the formulation approved by government standards. For example, the following compounds: benzoic acid, sorbic acid, phenoxyethanol, methylparaben, chlorocresol, benzalkonium chloride, and cetrimide, are strongly active in fighting off the growth of gram-positive and gram-negative bacteria, however, they are slightly weaker in opposing the growth of molds and yeasts. Accordingly, these preservatives may advantageously be used in a concentration ranging from about 0.001% to about 0.8% [weight to volume] to effectively preserve the methadone formulation. Simple alcohol, such as ethanol, is useful as a preservative when it is used as a solvent. However, a relatively high concentration, somewhat greater than about 10%, is needed to be effective. More organic alcohols, such as phenethyl alcohol, are useful in lower concentrations, such as about 1% for preservative action. Acids, such as benzoic acid and sorbic acid have lower solubility in water, and may be used for inhibitory action in a concentration ranging from about 0.1% to about 0.5% w/v. Only the non-ionized form of the acid is effective and, therefore, its use is restricted to preparations with a pH below about 4.5. Esters, such as methyl, propyl, and butyl esters of paraben (p-hydroxybenzoic acid) generally have decreasing water solubility as the molecular weight of the paraben, or its alkoxide ester group, increases. For example, solubility of about 0.25% for the methyl ester decreases to about 0.2% for the butyl ester. These compounds are effective as preservatives and stable over a pH range of about 3 to about 9.5. They are typically utilized at concentrations up to about 0.8% w/v. Frequently, two paraben esters may be used in combination in the same preparation. This achieves a higher total concentration, and the mixture tends to be more active against a wide range of microorganisms. However, it should be noted that paraben activity is reduced in the presence of non-ionic surface-active agents due to binding. In addition, hydrolytic decomposition of the ester group occurs with a loss of activity in ionic alkaline solutions. Certain quaternary ammonium compounds, such as benzalkonium chloride, are preservatives used at relatively low concentrations such as about 0.01% to about 0.25% w/v, depending on the nature of the pharmaceutical product. This class of compounds has optimal activity over the pH range from about 4 to about 10 and is quite stable at room temperature. Due to the cationic nature of this type of preservative, it is incompatible with many anionic compounds such as surfactants. To this end, it is generally used for the preparation of formulations intended for external use, such as topical application onto a mucous membrane.

The following tables will further illustrate concentration ranges of exemplary preservatives that may be included in the present compositions.

TABLE 1
Microbiological and physiochemical
properties of selected preservatives

| | In-use | | |
|---|---|---|---|
| Preservative | concentration(w/v %) | pH range^{a} | O/W^{b} |
| Benzoic acid | 0.1 | 2-5 | 3-6 |
| Sorbic acid | 0.2 | <6.5 | 3.5 |
| Phenoxyethanol | 0.5-1.0 | wide | — |
| Benzylalcohol | 0.75-5.0 | — | — |
| Methylparaben | 0.05-0.8 | 3.0-9.5 | 7.5 |
| Propylparaben | 0.01-0.8 | 3.0-9.5 | 80 |
| Butylparaben | 0.4-0.8 | 3.0-9.5 | 280 |
| Phenol | 0.2-0.5 | — | — |
| Meta-cresol | 0.1-0.3 | — | — |
| Chlorocresol | 0.1 | <8.5 | 117-190 |
| Benzalkonium chloride | 0.01-0.25 | 4-10 | <1 |
| Benzethonium chloride | 0.01 | — | — |
| Cetrimide | 0.01-0.1 | 4-10 | <1 |
| Phenyl mercuric nitrate | 0.001 | — | — |
| Thimersol | 0.003-0.01 | — | — |
| Myristyl gamma- | 0.0195-0.169 | — | — |
| picolinium chloride | | | |

^{a}Optimal pH range for activity
^{b}Oil-water partition coefficient

TABLE 2
Regulatory approved ophthalmic antimicrobial preservatives

| Antimicrobial preservative | typical concentration range (w/v %) |
|---|---|
| Benzalkonium chloride | 0.01-0.02 |
| Benzethonium chloride | 0.01-0.02 |
| Chlorhexidine | 0.002-0.01 |
| Methylparaben | 0.015-0.05 |
| Phenylethyl alcohol | up to 0.5 |
| Phenylmercuric acetate | 0.001-0.002 |
| Phenylmercuric borate | 0.002-0.004 |
| Phenylmercuric nitrate | 0.002-0.004 |
| Propyl paraben | 0.005-0.01 |
| Thimersol | 0.001-0.15 |

Additional information regarding the selection and concentration of government approved preservatives, suitable for use in the present invention, is described in Pharmaceutical Preformulation and Formulation: A Practical Guide from Candidate Drug Selection to Commercial Dosage Form, HIS Health group publication, 2001, and The Science and Pharmacy, Mack publishing Co, 1995, the disclosures of which are incorporated by reference herein in their entireties.

The present chlorobutanol-free, methadone-containing compositions, either in a sterile formulation and/or including a chlorobutanol-free preservative, are formulated into a form approved for parenteral administration. To this end, the formulation may generally be a liquid or a solid. The most common liquid formulations are solutions. Solutions are homogeneous mixtures that are prepared by dissolving a solid, liquid, or gas into a diluent, usually another liquid, and represents a group of preparations in which the molecules of the solute or dissolved substance are dispersed among those of the solvent. Other formulations include semi-solids, such as suspensions, gels, creams, pastes, emulsions, and the like, typically prepared with a diluent. Water is typically used as the diluent (solvent) or the major ingredient in liquid and semi-solid formulations. Water serves both as a vehicle and as a solvent for the desired formulation of medicinal ingredients. Its tastelessness, freedom from irritating qualities, and lack of pharmacological activity make it ideal for such purposes. Water used for pharmaceutical formulations must meet the appropriate pharmacopoeia standard for the intended use.

The formulator must pay special care and attention to the preparation formed, particularly with pastes, emulsions, and suspension. For example, emulsions may cream, but if they break (i.e., there is a separation of an oil phase), the methadone formulation will be considered to be unstable. Sedimentation and caking are primary indications of instability in suspension. The presence of large particles may mean that excessive crystal growth has occurred. Such attention is important particularly at the time of administration. To this end, it is beneficial to formulate the composition in a manner so as to avoid such problems, and particularly, the formulation may be compounded just prior to or at the time of administration.

Thus, there is provided chlorobutanol-free compositions comprising methadone and a chlorobutanol-free preservative, and chlorobutanol-free compositions comprising methadone in sterile formulations, for parenteral administration to a patient. The present compositions and formulations eliminates the cardiotoxity that was previously associated with parenteral methadone therapy. This invention provides for methadone formulations free of cardiotoxicity and cardiac side effects and useful in treating pain and the emeleoriation of opiate withdrawl. To this end, the present invention provides the outstanding benefits and properties of methadone in a safe, parenterally administrable formulation. Particularly, the compositions of the present invention may effectively be used for the treatment of pain refractory attributable to a variety of pain, such as chronic pain, cancer pain, acute pain including neuropathic pain, autonomic nervous system pain, and central pain. The present invention may also be used to manage the side effects of opiate addiction or to manage the withdrawl from dependence on opiate analgesics.

While the present invention has been illustrated by the description of embodiments thereof, and while the embodiments have been described in considerable detail, it is not intended to restrict or in any way limit the scope of the appended claims to such detail. Additional advantages and modifications will be readily apparent to those skilled in the art. The invention in its broader aspects is therefore not limited to the specific details, representative method, and examples described. Accordingly, departures may be made from such details without departing from the scope or spirit of Applicant's general inventive concept.

What is claimed is:

1. A pharmaceutical composition comprising methadone and a biologically acceptable, chlorobutanol-free preservative in a parenterally administrable formulation.

2. The composition of claim 1 wherein the preservative is present in an amount that is non-toxic.

3. The composition of claim 1 wherein the preservative is government approved.

4. The composition of claim 1 wherein the preservative is a compound selected from the group consisting of a paraben, an aromatic alcohol, a cresol, an acetate, a borate, a nitrate, an acid, and a combination thereof.

5. The composition of claim 1 wherein the preservative is an antimicrobial preservative selected from the group consisting of benzalkonium chloride, benzethonium chloride, benzyl alcohol, phenethyl alcohol, phenoxyethanol, meta-cresol, chlorocresol, phenol, chlorhexidine, methylparaben, propylparaben, butylparaben, phenylmercuric acetate, phenylmercuric borate, phenylmercuric nitrate, thimerosal, benzoic acid, sorbic acid, centrimide, myristyl gamma-picolinium chloride, and a combination thereof.

6. The composition of claim 1 wherein the formulation is sterile.

7. The composition of claim 1 wherein the formulation is adapted for topical administration to a membrane.

8. The composition of claim 7 wherein the formulation further comprises a diluent, the methadone having a concentration in the range from about 0.1 mg per ml to about 20 mg per ml of the diluent.

9. The composition of claim 1 wherein the formulation is adapted for ocular administration to an eye.

10. The composition of claim 9 wherein the formulation further comprises a diluent, the methadone having a concentration in the range from about 0.01 mg per ml to about 10 mg per ml of the diluent.

11. The composition of claim 1 in a formulation selected from the group consisting of a solution, a paste, a powder, an emulsion, and a suspension.

12. The composition of claim 1 wherein the formulation further comprises a diluent, wherein the methadone has a concentration in the range from about 0.005 mg per ml to about 50 mg per ml of the diluent.

13. A pharmaceutical composition consisting essentially of methadone in a parenterally administrable sterile formulation.

14. The composition of claim 13 in a formulation selected from the group consisting of a solution, a paste, a powder, an emulsion, and a suspension.

15. The composition of claim 13 wherein the formulation is adapted for topical administration to a membrane.

16. The composition of claim 13 wherein the formulation is adapted for ocular administration to an eye.

17. The composition of claim 13 wherein the formulation is adapted for intravenous administration to a patient.

18. The composition of claim 14 wherein the formulation further comprises a diluent, wherein the methadone has a concentration in the range from about 0.005 mg per ml to about 50 mg per ml of the diluent.

19. A method of treating pain in a patient comprising parenterally administering to a patient a pharmaceutical composition comprising methadone and a biologically acceptable, chlorobutanol-free preservative, in an amount effective for treating the pain.

20. The method of claim 19 wherein the pharmaceutical composition comprising methadone in an amount effective for treating pathological itching.

21. The method of claim 19 further comprising formulating the composition into one of a solution, a paste, a powder, an emulsion, and a suspension, for parenteral administration to the patient.

22. The method of claim 21 further comprising, prior to administration, sterilizing the formulation by sterile filtration, irradiation, heat, autoclave, or combinations thereof.

23. The method of claim 19 wherein the pain is attributable to one of a cancer pain, a neuropathic pain, a chronic pain, an acute pain, a somatic pain, an autonomic nervous system mediated pain, a central pain, and a combination thereof.

24. The method of claim 19 wherein the preservative is government approved and present in the composition in an amount that is non-toxic.

25. The method of claim 19 wherein the preservative is a compound selected from the group consisting of a paraben, an aromatic alcohol, a cresol, an acetate, a borate, a nitrate, an acid, and a combination thereof.

26. The method of claim 19 wherein the preservative is an antimicrobial preservative selected from the group consisting of benzalkonium chloride, benzethonium chloride, benzyl alcohol, phenethyl alcohol, phenoxyethanol, meta-cresol, chlorocresol, phenol, chlorhexidine, methylparaben, propylparaben, butylparaben, phenylmercuric acetate, phenylmercuric borate, phenylmercuric nitrate, thimerosal, benzoic acid, sorbic acid, cetrimide, myristyl gamma-picolinium chloride, and a combination thereof.

27. The method of claim 21 wherein the formulation administered further comprises a diluent, the methadone formulated to have a concentration in the range from about 0.005 mg per ml to about 50 mg per ml of the diluent.

28. A method of treating pain in a patient comprising topically administering to a membrane of the patient a sterile, chlorobutanol-free formulation comprising methadone in an amount effective for treating the pain.

29. The method of claim 28 wherein the formulation administered is selected from the group consisting of a solution, a paste, a powder, an emulsion, and a suspension.

30. The method of claim 28 further comprising, prior to administration, sterilizing the formulation by sterile filtration, irradiation, heat, autoclave, or combinations thereof.

31. The method of claim 28 wherein the formulation is administered to a mucosal membrane selected from the group consisting of a nasal membrane, a buccal membrane, a vaginal membrane, a rectal membrane, and a combination thereof.

32. The method of claim 28 wherein the pain is attributable to one of a cancer pain, a chronic pain, an acute pain, a somatic pain, an autonomic nervous system mediated pain, a central pain and a combination thereof.

33. The method of claim 28 wherein the formulation administered further comprises a diluent, the methadone formulated to have a concentration in the range from about 0.005 mg per ml to about 20 mg per ml of the diluent.

34. A method of treating opthalmic pain in a patient comprising administering to an eye of the patient a chlorobutanol-free, pharmaceutical composition comprising methadone in an amount effective for treating the pain.

35. The method of claim 34 further comprising formulating the composition into one of a solution, a paste, a powder, an emulsion, and a suspension for ocular administration.

36. The method of claim 35 further comprising, prior to administration, sterilizing the formulation by sterile filtration, irradiation, heat, autoclave, or combinations thereof.

37. The method of claim 35 wherein the formulation administered further comprises a diluent, the methadone formulated to have a concentration in the range from about 0.01 mg per ml to about 10 mg per ml of the diluent.

38. The method of claim 35 wherein the composition further comprises a biologically acceptable, chlorobutanol-free preservative.

39. The method of claim 38 wherein the preservative is government approved and present in an amount that is non-toxic.

40. The method of claim 38 wherein the preservative is a compound selected from the group consisting of a paraben, an aromatic alcohol, a cresol, an acetate, a borate, a nitrate, an acid, and combinations thereof.

41. The method of claim 38 wherein the preservative is an antimicrobial preservative selected from the group consisting of benzalkonium chloride, benzethonium chloride, benzyl alcohol, phenethyl alcohol, phenoxyethanol, meta-cresol, chlorocresol, phenol, chlorhexidine, methylparaben, propylparaben, butylparaben, phenylmercuric acetate, phenylmercuric borate, phenylmercuric nitrate, thimerosal, benzoic acid, sorbic acid, cetrimide, myristyl gamma-picolinium chloride, and combinations thereof.

42. A method of treating opthalmic pain in a patient comprising administering to an eye of the patient a sterile, chlorobutanol-free formulation comprising methadone in an amount effective for treating pain therein.

43. The method of claim 42 wherein the formulation administered is one of a solution, a paste, a powder, an emulsion, and a suspension for ocular administration.

44. The method of claim 42 further comprising, prior to administration, sterilizing the formulation by sterile filtration, irradiation, heat, autoclave, or combinations thereof.

45. The method of claim 42 wherein the formulation further comprises a diluent, the methadone formulated to have a concentration in the range from about 0.01 mg per ml to about 10 mg per ml of the diluent.
